(19) United States
(12) Patent Application Publication
(10) Pub. No.: US 2005/0238678 A1
(43) Pub. Date: Oct. 27, 2005

(54) Hydrogels from biopolymers

(76) Inventors: Janos Borbely, Debrecen (HU); Eva Fleischer, Debrecen (HU); Levente Novak, Debrecen (HU); Zsuzsa Maria Borbely, Debrecen (HU)

Correspondence Address: Thomas A. O'Rourke;Bodner & O'Rourke, 425 Broadhollow Road, Melville, NY, 11747, US

(21) Appl. No.: 11/074,314

(22) Filed: Mar. 7, 2005

Related U.S. Application Data
(60) Provisional application No. 60/550,935, filed on Mar. 5, 2004.

(51) Int. Cl. C12P 21/06; A61K 7/00

(52) U.S. Cl. 424/401; 435/69.1

(57) ABSTRACT

Methods are disclosed for preparing linear or/and partial precross-linked poly-g-glutamic acid nanoparticle products, their reaction with compounds which contain vinyl groups, and the polymerization by chemical initiation or photopolymerization of these by light of predetermined wavelength. The final products of the present invention are useful as local drug delivery systems, dental surgery, and for inhibition of post-surgical adhesion. The hydrogels made from the biopolymers of the present invention may also be used in controlled release devices, superabsorbent materials and biomaterials like enzyme immobilization.

This is a conversion of Provisional Patent Application Ser. No. 60/550,935 filed Mar. 5, 2004 the disclosures of which are incorporated herein by reference.

### FIELD OF THE INVENTION

The present invention is directed to hdrogels made from poly-g-glutamic acid compounds that have been amidized with amine groups.

### BACKGROUND OF THE INVENTION

Biomaterials made from polymers are being extensively applied in medicine and biotechnology, as well as in other industries. Applications include use in surgical devices, implants and supporting materials (e.g. artificial organs, prostheses and sutures), drug-delivery systems with different routes of administration and design, carriers of immobilized enzymes and cells, biosensors, components of diagnostic assays, bioadhesives, ocular devices, and materials for orthopaedic applications.

Polymers used as biomaterials can be synthesized to have appropriate chemical, physical, interfacial and biomimetic characteristics, which permit various specific applications. Compared with other types of biomaterial, polymers offer the advantage that they can be prepared in different compositions with a wide variety of structures and properties. Interpenetrating polymer networks (IPNs), whose properties can be adjusted by varying their compositions, are an example of such a biomaterial. IPNs have a wide range of applications including drug delivery, soft-tissue replacement and vascular devices.

Polymers used as biomaterials can be naturally occurring, synthetic or a combination of both. Naturally derived polymers are abundant and usually biodegradable. Their principal disadvantage lies in the development of reproducible production methods, because their structural complexity often renders modification and purification difficult. Additionally, significant batch-to-batch variations occur because of their ‘biopreparation’ in living organisms (plants, crustaceans). Synthetic polymers are available in a wide variety of compositions with readily adjusted properties. Processing, copolymerization and blending provide simultaneous means of optimizing a polymer's mechanical characteristics and its diffusive and biological properties. The primary difficulty is the general lack of biocompatibility of the majority of synthetic materials, although poly(ethylene oxide) (PEO) and poly(lactic-co-glycolic acid) are exceptions.

The selection of polymers for matrices or carriers in controlled-delivery devices requires the consideration of characteristics such as molecular weight, adhesion and solubility, depending on the type of system to be prepared, its action and the target site in the body. For example, high molecular weight polymers cannot cross the blood-brain barrier and are not resorbed after oral administration. In some cases, polymeric materials for drug delivery must satisfy additional requirements, such as environmental responsiveness (e.g. pH− or temperature dependent phase or volume transformations). Hydrophilic and amphiphilic polymers are preferred for certain specialized applications (e.g. ophthalmological applications). In such cases, the polymers must have good bioadhesive properties (adhesive gels, membranes), which promote site-specific interactions between the material surface and cells. In many cases, the criteria for the design of polymeric systems depend on the target site for the action of the drug, as in ophthalmology.

Synthetic polymers have become more attractive owing to the potential for controlling their properties by tailoring their molecular structure. The use of chemically engineered synthetic polymers enables very precise manipulation of the physical characteristics and mechanical properties of the materials, porosity and degradation times. Biodegradable polymeric systems, which are completely bioresorbable in the body, are particularly attractive in tissue engineering. One of their main advantages is that they eliminate the need for surgical removal of a polymer matrix.

The use of biodegradable synthetic polymers as biomaterials is particularly attractive because their mechanical and physical properties can readily be adjusted by varying the preparation techniques and molecular structure. Copolymers based on poly(lactic acid) and poly(glycolic acid) also offer tuned degradability and are thus ideal for tissue-cell-seeded constructs. Hydrogels based on natural polymers (e.g. alginate, agarose and chitosan) have also been explored for the immobilization of a variety of mammalian cell types. Polyesters, polyanhydrides, polyamides and natural polymers, as well as networks, copolymers, blends and microcapsules based on these polymers, have been extensively studied and used as biodegradable matrices for the release of bioactive agents. The properties of these polymers can be tailored by combining this inorganic backbone with side-chain functionality. The resulting polymers can be hydrophilic, hydrophobic or amphiphilic, and can also be made into films, membranes and hydrogels for biomedical application by cross-linking or grafting.

Hydrogels are biocompatible owing to their high water-sorption capacity, which results in weak interactions with the extracellular-fluid components. Water adsorption also plays a key role in the strength, creep resistance and durability of biomaterials, which may be affected by hydrolytic degradation. Finally, water sorption is required in applications such as controlled drug delivery and sutures, where a regulated hydrolytic-degradation rate and optimum diffusion characteristics are desirable. In such cases, the surface has to be hydrophilic and possess different polar groups, usually derived from grafted polymers and hydrophilic polymer coatings. The water permeability of the materials is generally considered in the light of their solubility or swelling ability. A high water adsorption capacity corresponds to a high water permeability and high material hydrophilicity; hydrophilicity is not only related to the biocompatibility and functional performance of the material devices—it also determines other properties such as adhesion, environmental responsiveness to external aqueous stimuli (pH, glucose or urea) and degradation rate. Controlled changes in the swelling characteristics of the materials owing to environmental changes is useful in applications such as site-targeted drug delivery.

Bioactive compounds, such as enzymes, drugs, proteins, peptide sequences, antigens and cells, have been incorporated into polymeric materials in order to improve their biofunctionality and yield biologically active systems. Such modifications can have a significant influence on the biological response to biomaterials, which may also be used in bioreactors (e.g. immobilized enzymes), artificial organs and drug delivery systems.

Hydrogels and microcapsules are the most widely studied polymeric biomaterials. Hydrogels are synthesized by chemical or physical cross-linking, creating covalent, ionic or hydrogen bonds. Such structures have been obtained from natural and synthetic polymers and single-, double- and multiple-component polymer systems and have been applied to the immobilization of biomolecules and cells. Polymer hydrogels are most widely used for microencapsulation. Hydrogels are also used in wound dressings. Hydrogels act by autolysis, which is the breakdown of dead tissue by enzymes released by dead or damaged cells as part of a natural healing process, thereby hydrating the wound. When placed in contact with a wound the dressing absorbs exudates and produces a moist healing environment at the surface of the wound, without causing tissue maceration. Hydrogels are easily removed with water. Hydrogels can be used to both cleanse and protect contaminated wounds. They are indicated for use in shallow and deep open wounds e.g. pressure sores, lacerations and grazes. Hydrogels are of particular benefit in treating dry, sloughy or necrotic wounds, and for reaching wounds in awkward places as they are applied directly from the tube or applicator.

Various methods of preparing poly-γ-glutamic acid (PGA) and uses therefor are disclosed in the following U.S. Pat. No. 3,719,520 Fujimoto et al., U.S. Pat. No. 5,118,784 Kubota et al., U.S. Pat. No. 5,378,807 Gross et al., U.S. Pat. No. 5,461,085 Nagamoto et al., U.S. Pat. No. 5,545,681 Honkonen et al., U.S. Pat. No. 5,525,682 Nagatomo et al., U.S. Pat. No. 6,068,853 Giannos et al., U.S. Pat. No. 6,201,065 Pathak et al., U.S. Pat. No. 6,326,511 Borbely, the disclosures of which are incorporated herein by reference

### SUMMARY OF THE INVENTION

The present invention relates to hydrogels made from poly-γ-glutamic acid (PGA) compounds. Precross-linked poly-γ-glutamic acid (PGA) compounds are prepared by amidizing PGA with amine groups. In the preferred embodiment, the PGA first reacts with a diamino or polyamin compound forming a partially crosslinked nanoparticle. The surface of the PGA compound so formed is provided with a plurality of vinyl groups. Then the vinyl group undergos radical polymerization forming hydrogel. More particularly, after amidizing the poly-g-glutamic acid, the next step is the reaction of these precross-linked PGA compounds with one or more vinyl groups, and their polymerization by chemical initiation or polymerization preferably through the use of photopolymerization, i.e., upon exposure to light of a predetermined, specified wavelength, to form hydrogels. The preferred wavelength of the light is blue light. The preferred exposure is from about 1 minute to about 2.5 hours.

Where chemical initiation is performed instead of photpolymerization, the preferred initiators are potassium-persulphate or ammonium-persulphate, although others may be used as well. As a catalyst in the chemical initiation, TEMED (tetramethyl-ethylene-diamine), or DMAPN (3-dimethylamino-prophyonitrile) are preferred.

### DETAILED DESCRIPTION

The starting material of the present invention is PGA which is prepared by fermentation with a suitable microorganism, capable of producing PGA in a suitable fermentation medium, under conditions and time appropriate for the microorganism used. The resulting culture medium is treated, by centrifugation, to separate the cells from the PGA. The resulting cell-free liquid is treated with acetone to obtain the PGA from this fermentation medium. After obtaining the PGA from the fermentation medium, the PGA so obtained was purified by dialysis and subsequently freeze dried. The molecular weight of the PGA is typically about 1,000,000.

After freeze drying, the PGA is then partially amidated by reacting it with a diamino or polyamino compound. A preferred diamino compound is a linear di/tri/polyamines, such as:
NH₂—CH₂—CH₂—(O—CH₂—CH₂)n-NH₂ (EDBEA) where n=2 to 12
Other preferred diamino or polyamino compounds can include heterocyclic di/tri/polyamines, such as piperazine, aromatic di/tri/polyamines, such as 1,4-diphenyl amine, and heteroaromatic di/tri/polyamines, such as adenine. Other diamino or polyamino compounds can include one or more of the following or blends thereof:
1,3-diaminoacetone
2,4-diaminobutyric acid
1,3 -diaminoguanidine
1,3-diamino-2-propanol
   Cycloaliphatic di/tri/polyamine such as:
1,8-diamino-p-menthane
2,5-diazabicyclo[2.2.1]heptane
   Heterocyclic di/tri and polyamine, such as:
piperazine-2-carboxylic acid
   Aromatic di/tri/polyamine, such as:
2,5-diaminobenzenesulfonic acid
3,5-diaminobenzoic acid
   Heteroaromatic di/tri/polyamine, such as:
2,6-diaminopurine
2,3-diaminopyridine
2,5-diamonopyridine
2,6-diaminopyridine

The amidizing reaction that is performed determinates the precross-linking of the PGA. This precross-linking can performed so that there different amounts of crosslinking in the final product, i.e., from 1 to 99% cross linking. The amidizing reaction takes place in water, in the presence of a water soluble diimide compound, which preferably is dimethylamino propyl ethylcarbodiimide methiodide.

These partial precross-linked products can be further vinylized with different compounds if desired. These vinylizing compound include but are not limited to AEM (aminoethyl methacrylate hydrochloride) and other water soluble vinyl monomers containing amino functionality. The content of vinyl groups is preferably from about 5 to about 50%, more preferably 10 to 30%, reported to the free carboxyl groups from the precross-linked PGA products. Using these products (precross-linked and vinylized PGA) IPN hydrogels were obtained, using other soluble monomers, including but not limited to any one of the following or blends thereof:
acrylic acid
acrylic anhydride
acrylic acid anhydride
2-acrylamino-2-methyl-1-propanesulfonic acid
2-acryloxyethyltrimethylammonium chloride
N-acryloxysuccinimide
Bis(2-acryloxyehtyl)phosphate
2-carboxyethyl acrylate
glycerol monoacrylate
hydroxyethyl acrylate
hydroxypropyl acrylate
itaconic acid
monoacryloxyethyl phosphate
methacylic acid
methacrylic acid anhydride
cinnamyl methacrylate
glycerol monomethacrylate
hydroxyethyl methacrylate
hydroxypropyl methacrylate
methacryloyltris(hydroxymethyl)methylamine
N-methyl-N-vinylacetamiide
poly(ethyleneglycol)di/monomethacylate
poly(propyleneglycol)di/monomethacylate
N-vinyl-2-pyrrolidone
1-vinylimidazole
vinylsulphonic acid
N-vinyl urea
   The pH and ionic strength of the final hydrogel can be readily adjusted by changing the acidic, basic or neutral functions.

Alternatively, the formation of the hydrogels may be accomplished through the use of polymers which are water soluble but which do not react with the vinylized cross-linked PGA, and instead just penetrate its polymer network forming a SEMI-IPN(semi-interpenetrating polymer network).

The polymers which can be used to form a SEMI-IPN include but are not limited to the following:
Natural: polylisine, polyasparagine, chitosan, alginates, hyaluronic acid
Synthetic: polyacrylic/methacylic acid, poly-N-vinyl pyrolidone
   The final products of the present invention are useful in local drug delivery, and for inhibition of post-surgical adhesion. The products of the present invention may also be used in controlled release devices, superabsorbent materials and biomaterials like enzyme immobilization.

The following examples illustrate the present invention without, however, limiting the same thereto.

### EXAMPLES

### Example 1

### Preparation of Poly-g-Glutamic Acid

A solution was prepared by dissolving the following ingredients in 3 liter of distilled water.

| | |
|---|---|
| L-glutamic acid | 60 g |
| Citric acid | 78.8 g |
| Glycerol | 240 g |
| NH₄Cl | 21 g |
| K₂HPO₄ | 1.5 g |
| MgSO₄*7H₂O | 1.5 g |
| CaCl₂*2H₂O | 0.45 g |
| MnSO₄*H₂O | 0.24 g |
| FeSO₄*7H₂O | 0.14 g |

The pH was adjusted to 7.4 with NaOH. The medium was autoclaved.

A Bacillus licheniformis suspension was used to inoculate the flasks which contain the medium solution, and they were incubated on the shaker (150 rpm) for seven days, at 37 C. The contents of the culture flasks were centrifuged to separate the cells from the polymer solution. Two volumes of 99.5% acetone were added slowly to the supernatant liquid while stirring. The liquid was decanted and the precipitated polymer was dissolved in distilled water. The resulting polymer solution was dialyzed 1 day against EDTA solution, and 6 days against distilled water, and freeze dried.

### Example 2

### Partial Cross-Linking of PGA (10% of the Free Carboxyl Groups are Reacting)

To a 10 g/l aqueous solution of 0.2 g of the PGA from Example 1, 0.0433 CDI was added, and stirred 30 minutes. To the resulting solution 11.32 ml EDBEA was added, and stirred at ambient temperature for 24 hours. After this time the resulting polymer solution was dialyzed 7 days against distillated water, and freeze dried.

### Example 3

### Partial Cross-Linking of PGA (50% of the Free Carboxyl Groups are Reacting)

To a 10 g/l aqueous solution of 0.2 g of the PGA from Example 1, 0.2164 CDI was added, and stirred 30 minutes. To the resulting solution 56.6 μl EDBEA was added, and stirred at ambient temperature for 24 hours. After this time the resulting polymer solution was dialyzed 7 days against distillated water, and freeze dried.

### Example 4

### Reaction Between PGA and Products Which Contain Vinyl Group

To a 10 g/l aqueous solution of 0.2g of the PGA from Example 1, 0.2164 CDI was added, and stirred 30 minutes. To the resulting solution 0.1284 g AEM was added, and stirred at ambient temperature for 24 hours. After this time the resulting polymer solution was dialyzed 7 days against distillated water, and freeze dried.

### Example 5

### Reaction Between 50% Precross-Linked PGA and Products Which Contain Vinyl Goup

To a 10 g/l aqueous solution of 1 g of the 50% cross-linked PGA from Example 3, 0.2705 CDI was added, and stirred 30 minutes. To the resulting solution 0.1605 g AEM was added, and stirred at ambient temperature for 24 hours. After this time the resulting polymer solution was dialyzed 7 days against distillated water, and freeze dried.

### Example 6

### Photopolyimerization of 10% Cross-Linked PGA and N-vinyl-1-pirrolidone

To 210 mg precross-linked PGA from example 2 in 1.5 cm³ water, was added 90 mg N-vinyl-1-pirrolidone and 100 μl 1% photoinitiator (sodium antraquinone-2-sulphonate). The photopolimerization was made upon exposure to blue light with 350 nm wavelength for 10 minutes and sample solidifies as a hydrogel.

### Example 7

### Polymerization with Chemical Initiation of 50% Cross-Linked PGA

To 300 mg precross-linked PGA from example 5 in 1.5 ml water, was added 0.5 ml ammonium-persulphate (40 mg/ml) and 15 μl TEMED. The sample solidifies in 30 minutes as hydrogel.

### Example 8

### Polymerization with Chemical Initiation of 50% Cross-Linked PGA and PAA

To 250 mg precross-linked PGA from example 5 in 1.5 ml water, was added 50 mg PAA (poly acrylic acid), 0.5 ml ammonium persulphate (40 mg/ml) and 15 μl TEMED. The sample solidifies in 30 minutes as hydrogel.

### Example 9

### Polymerization with Chemical Initiation of 50% Cross-Linked PGA and N-vinyl-1-pirrolidone

To 200 mg precross-linked PGA from example 5 in 1.5 ml water, was added 100 mg N-vinyl-1-pirrolidone, 0.5 ml ammonium persulphate (40 mg/ml) and 15 μl TEMED. The sample solidifies in 10 minutes as hydrogel

1. A method of forming a hydrogel comprising: reacting a poly-γ-glutamic acid with a diamino or polyamino compound; reacting the resulting product to blue light with 350 nm wavelength in the presence of a photoinitiator

2. The method according to claim 1 wherein the diamino or polyamino compound is N-vinyl-1-pirrolidone.

3. The method according to claim 1 wherein the photoinitiator is sodium antraquinone-2-sulphonate).

4. The method according to claim 1 wherein the poly-y-glutamic acid is prepared by forming a medium by dissolving in distilled water:

| | |
|---|---|
| L-glutamic acid | 10-18% by weight |
| Citric acid | 17-23% by weight |
| Glycerol | 55-65% by weight |
| NH₄Cl | 3-7% by weight |
| K₂HPO₄ | 0-.5% by weight |
| MgSO₄*7H₂O | 0-.5% by weight |
| CaCl₂*2H₂O | 0-.3% by weight |
| MnSO₄*H₂O | 0-.3% by weight |
| FeSO₄*7H₂O | 0-.3% by weight |

Adjusting the pH to about 7.4;
Innoculating a receptacle containing the medium with Bacillus licheniformis and incubating the medium; separating a polymer solution so formed, dialyzing the resulting polymer solution, and freeze drying.

5. The method according to claim 1 wherein said poly-γ-glutamic acid is reacted with EDBEA.

6. The method according to claim 1 wherein said poly-γ-glutamic acid is reacted with CDI and EDBEA

7. The method according to claim 1 wherein said poly-γ-glutamic acid is reacted with AEM.

8. A method of forming a hydrogel comprising:
reacting a poly-γ-glutamic acid with a diamino or polyamino compound and reacting the resulting product with a chemical initiator in the presence of a catalyst to crosslink the product.

9. The method according to claim 8 wherein the chemical initiator is potassium-persulphate.

10. The method according to claim 8 wherein the chemical initiator is ammonium-persulphate.

11. The method according to claim 8 wherein the catalyst is TEMED (tetramethyl-ethylene-diamine).

12. The method according to claim 8 wherein the catalyst is DMAPN (3-dimethylamino-prophyonitrile).
